(19) United States
(12) Patent Application Publication
(10) Pub. No.: US 2024/0057850 A1
(43) Pub. Date: Feb. 22, 2024

(54) SCOPE DISTAL END ENCAPSULATION

(71) Applicant: BOSTON SCIENTIFIC SCIMED, INC., Maple Grove, MN (US)

(72) Inventors: Mark David Mirigian, Kilkenny (IE); Liam Ryan, Dungarvan (IE); Robert Hannon, Tipperary (IE); James Michael English, Cahir (IE); Barbara Belisa Soffiati, Clonmel (IE); Shane McGrath, Thurles (IE)

(73) Assignee: BOSTON SCIENTIFIC SCIMED, INC., Maple Grove, MN (US)

(21) Appl. No.: 18/452,014

(22) Filed: Aug. 18, 2023

Related U.S. Application Data
(60) Provisional application No. 63/399,552, filed on Aug. 19, 2022.

(51) Int. Cl. A61B 1/00; A61B 1/05; A61B 1/06

(52) U.S. Cl. CPC A61B 1/00096; A61B 1/0011; A61B 1/051; A61B 1/0676; A61B 1/0684

(57) ABSTRACT

The present disclosure provides to encapsulate the distal components of a scope to form a distal cap assembly, which removes the need for adhesives and potting often used in conventional distal cap assemblies. In particular, the present disclosure provides a spline structure configured to hold the distal end components in a predefined arrangement. The spline structure, along with the distal end components, is inserted into a mold and the spline structure along with the distal end components is encapsulated (e.g., in a polymer using hot melt, or the like) to form a distal cap. The distal cap locks the distal end components into the spline structure and protects the distal end components from moisture and relative movement. As a benefit, the present encapsulation techniques and distal cap assembly is faster to manufacture and provides less room for error than conventional methods.

### CROSS-REFERENCE TO RELATED APPLICATIONS

This application claims the benefit of U.S. Prov. Pat. App. No. 63/399,552, filed Aug. 19, 2022, titled SCOPE DISTAL END ENCAPSULATION, which is incorporated herein by reference.

### TECHNICAL FIELD

The present disclosure generally relates to the distal end of a scope. Particularly, but not exclusively, the present disclosure relates to encapsulating components at the distal end of a scope, such as, an endoscope.

### BACKGROUND

Modern endoscopes include a number of components (e.g., lights, camera, etc.) at the distal end. Often, these components are packaged or fit into a distal cap assembly. Typical distal cap assemblies are manufactured using a plastic molded cap, in which all the distal components are inserted into. Once inserted, the components are locked in place using adhesives and potting. This increases the chance of manufacturing defects due to the complexity and the size of the distal cap assemblies and the number of individual components. Further, the manufacturing time is typically increased due to the adhesive curing times. Thus, there is a need for an improved distal cap assembly.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides a method of manufacturing a distal cap assembly for a scope. The method can comprise providing a spline structure; fitting a plurality of distal end components into the spline structure; inserting the spline structure and the plurality of distal end components into a mold; and injecting encapsulation material into the mold to encapsulate the spline structure and the distal end components to form a distal end capsule.

In some embodiments, the method provides, wherein the spline structure comprises a plurality of channels, each one of the plurality of channels configured to receive a respective one of the plurality of distal end components. With some embodiments, the method provides, wherein the spline structure comprises at least one lumen connecting a proximal end of the spline structure to the plurality of channels. In some embodiments, the method provides, wherein at least one of the plurality of distal end components is an image sensor. With some embodiments, the method provides, wherein the at least one of the plurality of distal end components comprises a lead routed through the at least one lumen and coupled to the image sensor. In some embodiments, the method provides, wherein at least another one of the plurality of distal end components is a light emitting diode (LED). With some embodiments, the method provides, wherein the at least one other of the plurality of distal end components comprises a lead routed through the at least one lumen and coupled to the LED and wherein the encapsulation material is at least partially transmissive to a wavelength of light emitted by the light emitting diode. In some embodiments, the method provides, wherein the image sensor is a charge couple device (CCD) sensor or a complimentary metal-oxide semiconductor (CMOS) sensor. With some embodiments, the method comprises printing, depositing, or printing and depositing a plurality of layers of a spline structure material with a three-dimensional (3D) printer to form the spline structure. In some embodiments, the method provides, wherein the spline structure comprises a raised ring disposed between the proximal end of the spline structure and a distal end of the spline structure. With some embodiments, the method provides, wherein the distal end capsule entirely encapsulates at least one of the plurality of distal end components. In some embodiments, the method provides, wherein a distal most end of at least one of the distal end components is flush with a distal most end of the distal end capsule. With some embodiments, the method provides, wherein the encapsulation material is polymer. In some embodiments, the method provides, wherein injecting encapsulation material into the mold is part of a hot melt process.

The disclosure further provides a distal cap assembly. In some implementations, the distal cap assembly is manufactured according to the methods described herein. With some embodiments, the disclosure provides the distal cap assembly, comprising: a spline structure; a plurality of distal end components disposed in the spline structure; and a distal end capsule encapsulating the spline structure and the distal end components, wherein the distal end capsule is formed with an encapsulation material using a hot melt injection process.

In some embodiments, the distal cap assembly provides, wherein the spline structure comprises a plurality of channels, each one of the plurality of channels configured to receive a respective one of the plurality of distal end components. With some embodiments, the distal cap assembly provides, wherein the spline structure comprises at least one lumen connecting a proximal end of the spline structure to the plurality of channels. In some embodiments, the distal cap assembly provides, wherein at least one of the plurality of distal end components is an image sensor. With some embodiments, the distal cap assembly provides, wherein the at least one of the plurality of distal end components comprises a lead routed through the at least one lumen and coupled to the image sensor. In some embodiments, the distal cap assembly provides, wherein at least another one of the plurality of distal end components is a light emitting diode (LED). With some embodiments, the distal cap assembly provides, wherein the at least one other of the plurality of distal end components comprises a lead routed through the at least one lumen and coupled to the LED and wherein the encapsulation material is at least partially transmissive to a wavelength of light emitted by the light emitting diode. In some embodiments, the distal cap assembly provides, wherein the image sensor is a charge couple device (CCD) sensor or a complimentary metal-oxide semiconductor (CMOS) sensor. With some embodiments, the distal cap assembly provides, wherein the spline structure comprises a raised ring disposed between the proximal end of the spline structure and a distal end of the spline structure. In some embodiments, the distal cap assembly provides, wherein the distal end capsule entirely encapsulates at least one of the plurality of distal end components. With some embodiments, the distal cap assembly provides, wherein a distal most end of at least one of the distal end components is flush with a distal most end of the distal end capsule. In some embodiments, the distal cap assembly provides, wherein the encapsulation material is polymer.

The disclosure provides an endoscope comprising: a proximal end comprising at least one electric connector; a distal end comprising a plurality of distal end components; an outer lumen coupling the proximal end and the distal end; at least one lead disposed in the outer lumen, the at least one lead to electrically couple the at least one electric connector with the plurality of distal end components; wherein the distal end comprises a cap assembly, comprising: a spline structure, wherein the plurality of distal end components are disposed in the spline structure; and a distal end capsule encapsulating the spline structure and the distal end components, wherein the distal end capsule is formed with an encapsulation material using a hot melt injection process.

In some embodiments, the endoscope provides, wherein the spline structure comprises a plurality of channels, each one of the plurality of channels configured to receive a respective one of the plurality of distal end components. With some embodiments, the endoscope provides, wherein the spline structure comprises at least one distal lumen connecting a proximal end of the spline structure to the plurality of channels. In some embodiments, the endoscope provides, wherein at least one of the plurality of distal end components is an image sensor and wherein the at least one lead is routed through the at least one distal lumen; and wherein at least another one of the plurality of distal end components is a light emitting diode (LED) and wherein the encapsulation material is at least partially transmissive to a wavelength of light emitted by the light emitting diode. With some embodiments, the endoscope provides, wherein the spline structure comprises a raised ring disposed between a proximal end of the spline structure and a distal end of the spline structure and wherein the outer lumen abuts a first side of the raised ring proximate to the proximal end of the spline structure and wherein the distal end capsule abuts a second side of the raised ring proximate to the distal end of the spline structure. In some embodiments, the endoscope provides, wherein the distal end capsule entirely encapsulates at least one of the plurality of distal end components and wherein a distal most end of at least one of the distal end components is flush with a distal most end of the distal end capsule.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an aspect of the subject matter in accordance with one embodiment.

FIG. 2 illustrates an aspect of the subject matter in accordance with one embodiment.

FIG. 3 illustrates an aspect of the subject matter in accordance with one embodiment.

FIG. 4 illustrates an aspect of the subject matter in accordance with one embodiment.

FIG. 5A illustrates an aspect of the subject matter in accordance with one embodiment.

FIG. 5B illustrates an aspect of the subject matter in accordance with one embodiment.

FIG. 5C illustrates an aspect of the subject matter in accordance with one embodiment.

FIG. 6A illustrates an aspect of the subject matter in accordance with one embodiment.

FIG. 6B illustrates an aspect of the subject matter in accordance with one embodiment.

FIG. 6C illustrates an aspect of the subject matter in accordance with one embodiment.

FIG. 6D illustrates an aspect of the subject matter in accordance with one embodiment.

FIG. 6E illustrates an aspect of the subject matter in accordance with one embodiment.

### DETAILED DESCRIPTION

The present disclosure provides to encapsulate the distal components of a scope to form a distal cap assembly, which removes the need for adhesives and potting often used in conventional distal cap assemblies. In particular, the present disclosure provides a spline structure configured to hold the distal end components in a predefined arrangement. The spline structure, along with the distal end components, is inserted into a mold and the spline structure along with the distal end components is encapsulated (e.g., in a polymer using hot melt, or the like) to form a distal cap. The distal cap locks the distal end components into the spline structure and protects the distal end components from moisture and relative movement. As a benefit, the present encapsulation techniques and distal cap assembly is faster to manufacture and provides less room for error than conventional methods.

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

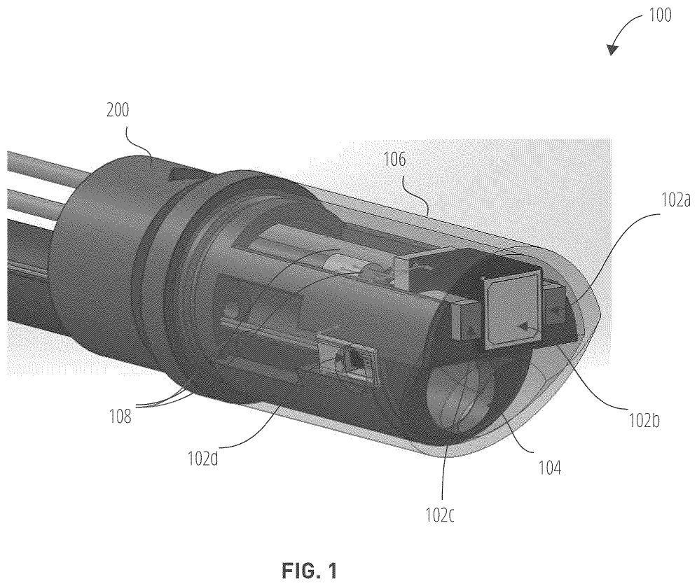

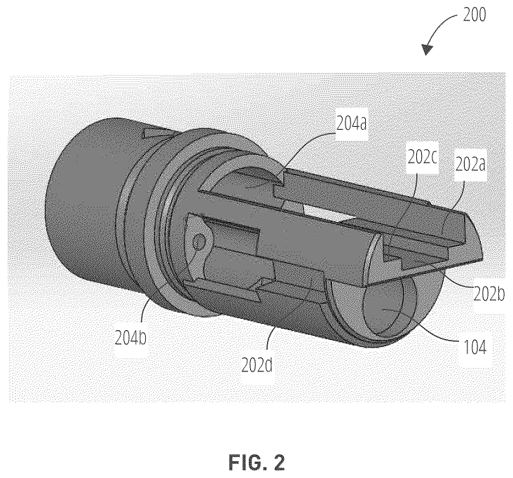

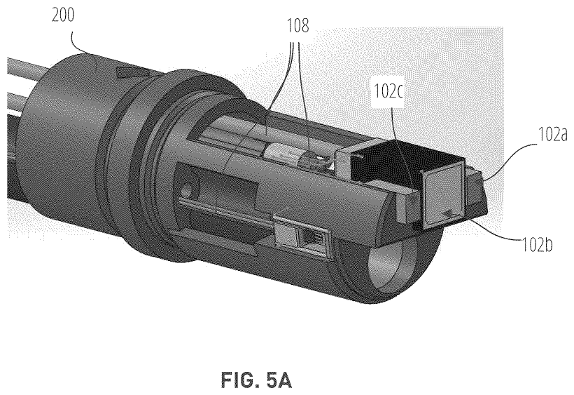

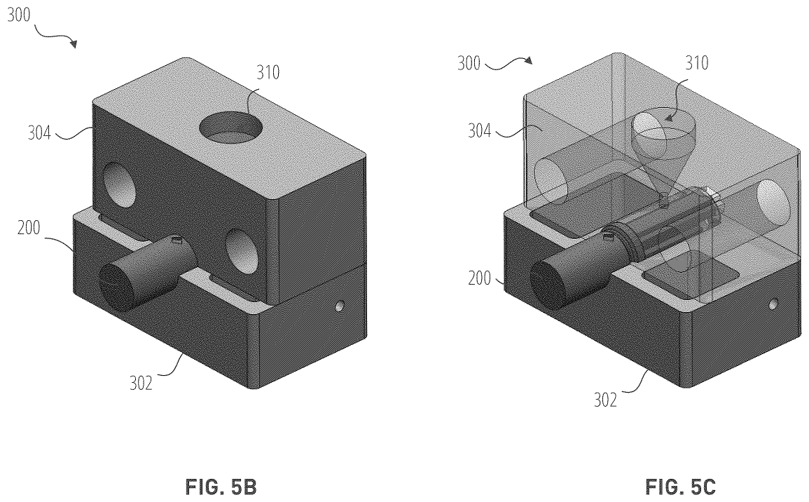

FIG. 1 illustrates a completed distal cap assembly 100, according to some embodiments of the present disclosure. In general, distal cap assembly 100 can form the distal end of any of a variety of endoscopes, such as, for example, a gastroscope, a colonoscope, a bronchoscope, a laryngoscope, a cystoscope, a duodenoscope, an enteroscope, a ureteroscope, a hysteroscope, or the like. As depicted, the distal cap assembly 100 of FIG. 1 shows a spline structure 200 (see FIG. 2) with various components disposed in the spline structure 200 (see FIG. 5A) and encapsulated as described herein. In particular, distal cap assembly 100 includes a number of distal end components 102a, 102b, 104, 102c, and 102d disposed in spline structure 200 and encapsulated in a material forming distal end capsule 106. In such examples, the distal end components 102a to 102d can be any of a variety of components that may include any combination of a camera, a lighting element (e.g., fiber optic cable, light emitting diode (LED), or the like), sensing devices, etc. Further, distal cap assembly 100 includes a working channel 104. It is noted that although distal cap assembly 100 only depicts a single working channel 104, multiple working channels 104 can be provided.

Some of the distal end components (e.g., 102a, etc.) can have electrical connections 108 coupled to the distal end components routed to a proximate end of the scope (not shown). The electrical connections 108 can be eventually coupled to electronics in the scope for which the spline structure 200 and distal cap assembly 100 are to be provided or coupled to hubs or further electrical connector at the proximal end of the scope and configured to provide electrical connection between a controller (e.g., image acquisition unit, computer, etc.) and the distal end components (e.g., distal end component 102a, etc.)

Distal cap assembly 100 can be sized based on the type of scope with which the distal cap assembly 100 is paired. For example, distal cap assembly 100 can have an outer diameter of 2 millimeters (mm), 15 mm, between 2 mm and 15 mm, or other sizes that may be suitable with use of a scope as described herein.

FIG. 2 illustrates spline structure 200, according to some embodiments of the present disclosure. As can be seen, spline structure 200 includes component channels 202a, 202b, 202c, and 202d, often referred to simply as “channels”. Spline structure 200 further includes component access ports 204a and 204b as well as working channel 104. Component channels 202a, 202b, 202c, and 202d can be configured and arranged to hold specific ones of the distal end components 102a, 102b, 102c, and 102d. For example, wherein distal end component 102b is a camera, component channel 202b can be configured and arranged to fit the dimensions of the camera and hold the camera securely during the encapsulation process (described in greater detail below). As another example, where distal end components 102a and 102c are LEDs, component channels 202a and 202c can be configured and arranged to fit the dimensions of the LEDs and hold the LEDs securely during the encapsulation process.

It is noted that although the component channels 202a, 202b, 202c, and 202d are depicted as square or rectangular shaped they can be any shape or size configured to hold the respective distal end components 102a, 102b, 102c, and 102d. For example if ones of the distal end components (e.g., distal end components 102a and/or 102b, or the like) are a fiber optic cable, the associated component channel(s) (e.g., component channels 202a and/or 202b) can be circular or oval shaped to securely hold the fiber optic cables during the encapsulation process.

Spline structure 200 further includes component access ports 204a and 204b. In general, component access ports 204a and 204b can be arranged to allow electrical connection (e.g., signal wires, conductors, cables, etc.) from the distal end components to be routed through the spline structure 200. As noted, these electrical connections (e.g., electrical connection 108) can be eventually coupled to electronics in the scope for which the spline structure 200 and distal cap assembly 100 are to be provided or coupled to hubs or electrical connections at the proximal end of the scope and configured to provide electrical connection between a controller (e.g., image acquisition unit, computer, etc.) and the distal end components.

It is important to note that the spline structure 200 includes channels (e.g., 202a, 202b, 202c, and 202d) for each respective distal end component to be encapsulated in the distal cap assembly 100. Further, the channels are shaped to hold the distal end components in position relative to each other during the encapsulation process without needing potting or other securing material or mechanisms. Further still, the spline structure 200 and channels provide that the distal end components are held in place (e.g., without adhesive and/or potting) while being inserted into a mold to encapsulation as described herein. This provides an advantage to conventional distal cap assemblies in that distal end components are retained in place (e.g., relative to each other, relative to the distal cap assembly, or the like) with less material and processing. As such, manufacturing times can be shortened and the cost of the manufacturing process reduced.

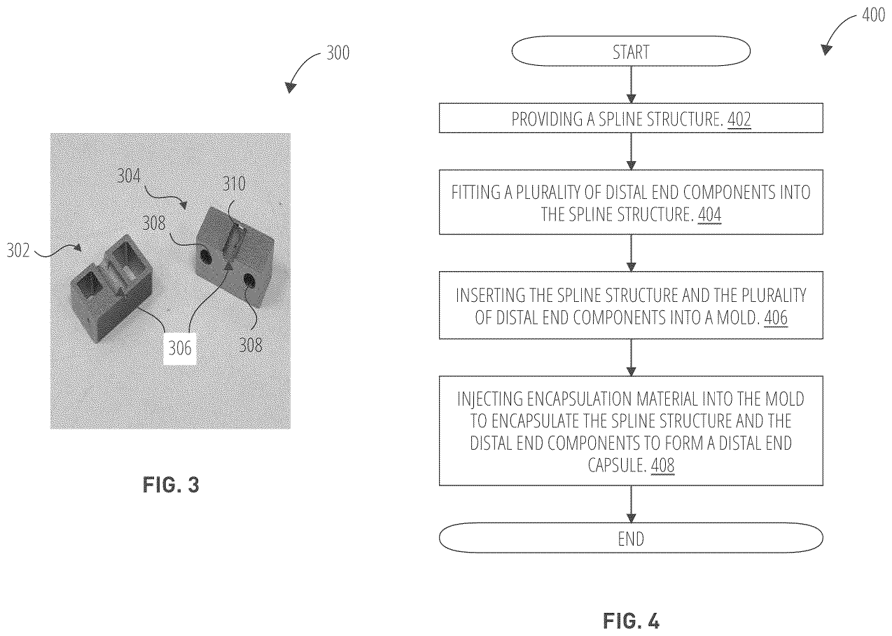

FIG. 3 illustrates a mold 300, according to some embodiments of the present disclosure. Mold 300 includes lower half 302 and upper half 304 and is arranged and configured for spline structure 200 to be inserted into a cavity 306 formed when the lower half 302 and upper half 304 are positioned proximate to each other such that the cavities 306 for a larger cavity in which spline structure 200 can be inserted, disposed, positioned, or fit.

Mold 300 further includes support structures 308, which can be configured to receive clamps, or other support structure to hold mold 300 (e.g., lower half 302 and upper half 304) in a stable position during the injection process. Additionally, mold 300 includes injection port 310 in which encapsulate material can be injected to surround and encapsulate the spline structure 200 and distal end components (e.g., 102a, etc.) to form the distal end capsule 106.

FIG. 4 illustrates a method 400, according to some embodiments of the present disclosure. The method 400 can be implemented to form distal end capsule 106 as outlined herein. In general, method 400 can be implemented to form any distal end capsule. However, for convenience and clarity, method 400 is described with reference to the distal cap assembly 100 of FIG. 1, spline structure 200 of FIG. 2, and mold 300 of FIG. 3. However, this is not intended to be limiting.

Method 400 can begin at block 402. At block 402, “provide a spline structure,” a spline structure can be provided. For example, spline structure 200 can be provided. The provided spline structure will include component channels and at least one working channel. Continuing to block 404, “fit a plurality of distal end components into the spline structure,” a plurality of distal end components can be fit into the component channels of the spline structure. For example, distal end components 102a, 102b, 102c, and 102d can be fit into component channels 202a, 202b, 202c, and 202d respectively.

This is more clearly illustrated in FIG. 5A, which shows the spline structure 200 with distal end components “fit” into the plurality of channels of the spline structure 200. For example, distal end components 102a, 102b, 102c, and 102d are depicted fit or positioned in respective ones of the component channels 202a, 202b, 202c, and 202d (not called out in this figure for clarity). Likewise, electrical connections 108 are depicted coupled to the distal end components and routed through the respective component access ports 204a and 204b (not called out in this figure for clarity).

Continuing to block 406, “insert the spline structure and the plurality of distal end components into a mold,” the spline structure can be inserted into a mold. For example, spline structure 200, including the distal end components 102a, can be inserted into the mold 300. This is more clearly illustrated in FIG. 5B and FIG. 5C, which shows spline structure 200 inserted into mold 300.

FIG. 5C illustrates a see-though version of the upper half 304 of mold 300 wherein the. Injection port 310 is depicted in greater detail. Injection port 310 can be arranged and configured to receive an encapsulate injector (described in greater detail below) with which encapsulate material can be injected into the cavity 306 to surround spline structure 200 and distal end components 102a, etc., such that the distal end capsule 106 is formed.

Continuing to block 408, “inject encapsulation material into the mold to encapsulate the spline structure and the distal end components to form a distal end capsule,” encapsulate is injected into the cavity of the mold to encapsulate the spline structure and the distal end components to form a distal end capsule. For example, encapsulate material can be injected into cavity 306, through injection port 310, to form distal end capsule 106 the surrounds and encapsulates spline structure 200 and distal end components 102a, 102b, 102c, and 102d. Further, the encapsulate material can form a distal or forward end of the scope for which the distal cap assembly 100 is configured, including a distal end of a working channel 104.

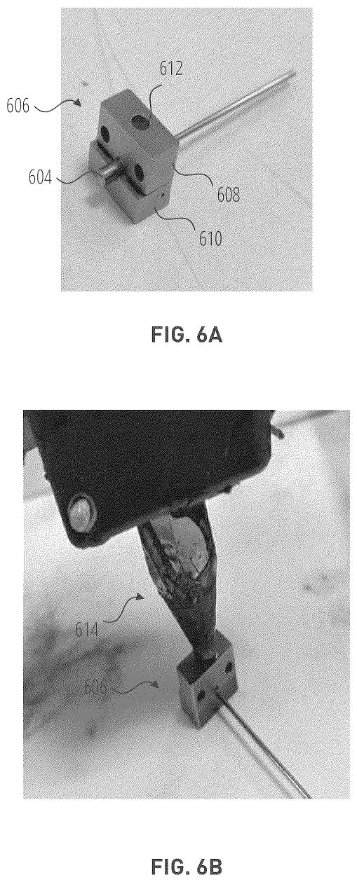

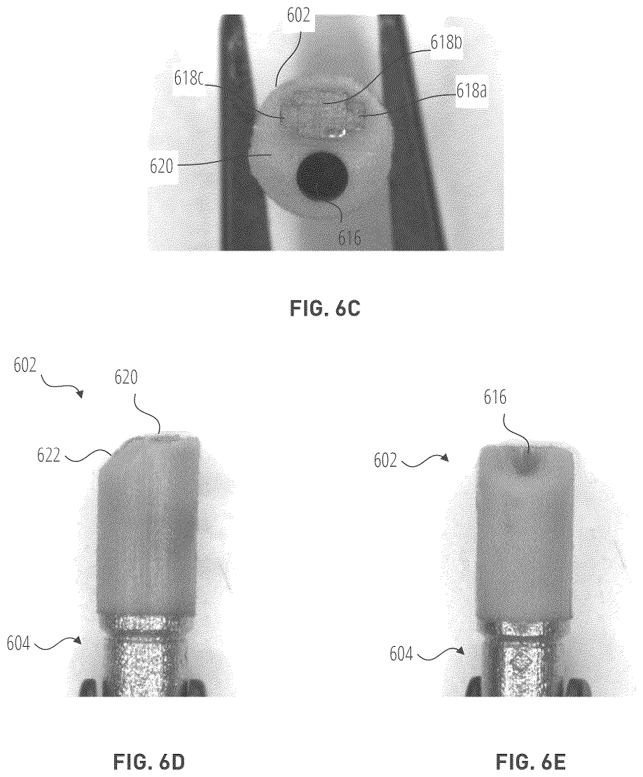

FIG. 6A through FIG. 6E illustrates a prototype distal end capsule 602 formed according to examples of the present disclosure. FIG. 6A illustrates a spline 604 inserted into mold 606. Mold 606 includes 608 and 610, which are depicted placed together to form a cavity (not shown) in which spline 604 is inserted. Further, the injection port 612 is shown in this figure.

FIG. 6B illustrates the mold 606 with an encapsulate injector 614 installed in the injection port 612 (obscured by encapsulate injector 614). Encapsulate material can be injected into the cavity to encapsulate and surround the spline 604 forming distal end capsule 602.

FIG. 6C illustrates a view from the distal end of the distal end capsule 602. As can be seen, the distal end capsule 602 includes a working channel 616 as well as distal end components 618a, 618b, and 618c.

FIG. 6D illustrates a side view of the distal end capsule 602 showing the distal end capsule 602 formed around the distal end of the spline 604, encapsulating the distal end components 618a, 618b, and 618c. As can be seen, the distal end capsule 602 can include a distal most face 620 and a sub-distal face 622. The sub-distal face 622 may slant slightly or be at an angle to the distal most face 620. For example, the sub-distal face 622 can be at an angle of between 25 and 50 degrees to the distal most face 620.

FIG. 6E illustrates another side view of the distal end capsule 602 showing the distal end capsule 602 formed around the distal end of the spline 604, encapsulating the distal end components 618a, 618b, and 618c (not shown) and the working channel 616.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A distal cap assembly, comprising:
a spline structure;
a plurality of distal end components disposed in the spline structure; and
a distal end capsule encapsulating the spline structure and the distal end components, wherein the distal end capsule is formed with an encapsulation material using a hot melt injection process.

2. The distal cap assembly of claim 1, wherein the spline structure comprises a plurality of channels, each one of the plurality of channels configured to receive a respective one of the plurality of distal end components.

3. The distal cap assembly of claim 2, wherein the spline structure comprises at least one lumen connecting a proximal end of the spline structure to the plurality of channels.

4. The distal cap assembly of claim 3, wherein at least one of the plurality of distal end components is an image sensor.

5. The distal cap assembly of claim 4, wherein the at least one of the plurality of distal end components comprises a lead routed through the at least one lumen and coupled to the image sensor.

6. The distal cap assembly of claim 5, wherein at least another one of the plurality of distal end components is a light emitting diode (LED).

7. The distal cap assembly of claim 6, wherein the at least one other of the plurality of distal end components comprises a lead routed through the at least one lumen and coupled to the LED and wherein the encapsulation material is at least partially transmissive to a wavelength of light emitted by the light emitting diode.

8. The distal cap assembly of claim 4, wherein the image sensor is a charge couple device (CCD) sensor or a complimentary metal-oxide semiconductor (CMOS) sensor.

9. The distal cap assembly of claim 1, wherein the spline structure comprises a raised ring disposed between the proximal end of the spline structure and a distal end of the spline structure.

10. The distal cap assembly of claim 1, wherein the distal end capsule entirely encapsulates at least one of the plurality of distal end components.

11. The distal cap assembly of claim 1, wherein a distal most end of at least one of the distal end components is flush with a distal most end of the distal end capsule.

12. The distal cap assembly of claim 1, wherein the encapsulation material is polymer.

13. An endoscope comprising:
a proximal end comprising at least one electric connector;
a distal end comprising a plurality of distal end components;
an outer lumen coupling the proximal end and the distal end;
at least one lead disposed in the outer lumen, the at least one lead to electrically couple the at least one electric connector with the plurality of distal end components;
wherein the distal end comprises a cap assembly, comprising:
a spline structure, wherein the plurality of distal end components are disposed in the spline structure; and
a distal end capsule encapsulating the spline structure and the distal end components, wherein the distal end capsule is formed with an encapsulation material using a hot melt injection process.

14. The endoscope of claim 13, wherein the spline structure comprises a plurality of channels, each one of the plurality of channels configured to receive a respective one of the plurality of distal end components.

15. The endoscope of claim 14, wherein the spline structure comprises at least one distal lumen connecting a proximal end of the spline structure to the plurality of channels.

16. The endoscope of claim 15, wherein:
at least one of the plurality of distal end components is an image sensor and wherein the at least one lead is routed through the at least one distal lumen; and
at least another one of the plurality of distal end components is a light emitting diode (LED) and wherein the encapsulation material is at least partially transmissive to a wavelength of light emitted by the light emitting diode.

17. The distal cap assembly of claim 1, wherein the spline structure comprises a raised ring disposed between a proximal end of the spline structure and a distal end of the spline structure and wherein the outer lumen abuts a first side of the raised ring proximate to the proximal end of the spline structure and wherein the distal end capsule abuts a second side of the raised ring proximate to the distal end of the spline structure.

18. The distal cap assembly of claim 1, wherein the distal end capsule entirely encapsulates at least one of the plurality of distal end components and wherein a distal most end of at least one of the distal end components is flush with a distal most end of the distal end capsule.

19. A method of manufacturing a distal cap assembly for a scope, comprising:
providing a spline structure;
fitting a plurality of distal end components into the spline structure;
inserting the spline structure and the plurality of distal end components into a mold; and
injecting encapsulation material into the mold to encapsulate the spline structure and the distal end components to form a distal end capsule.

20. The method of claim 19, comprising printing, depositing, or printing and depositing a plurality of layers of a spline structure material with a three-dimensional (3D) printer to form the spline structure.
